(19) United States
(12) Patent Application Publication
(10) Pub. No.: US 2015/0182424 A1
(43) Pub. Date: Jul. 2, 2015

(54) AEROSOL ANTIPERSPIRANT WITH REDUCED RESIDUE

(71) Applicant: The Dial Corporation, Scottsdale, AZ (US)

(72) Inventors: Catherine Schmit, Scottsdale, AZ (US); Noelle Rabiah, Scottsdale, AZ (US)

(21) Appl. No.: 14/141,625

(22) Filed: Dec. 27, 2013

(51) Int. Cl. A61K 8/04; A61Q 15/00; A61K 8/37

(52) U.S. Cl. CPC A61K 8/046; A61K 8/37; A61Q 15/00

(57) ABSTRACT

Materials and apparatus are provided for an aerosol antiperspirant composition with decreased residue transfer to fabric. The aerosol antiperspirant composition includes an active antiperspirant ingredient. The aerosol antiperspirant composition further includes a number of silicas. The aerosol antiperspirant composition further includes a polar emollient. The aerosol antiperspirant composition further includes a propellant. A manufacturing process for such an aerosol antiperspirant composition is also provided.

### FIELD OF THE INVENTION

The present invention generally relates to aerosol antiperspirants and more particularly relates to the use of polar emollients in aerosol antiperspirant compositions to decrease residue transfer to fabric.

### BACKGROUND OF THE INVENTION

An antiperspirant may be a composition which reduces the amount of perspiration on the skin to which the antiperspirant is applied. Aerosol antiperspirant products may be products which use a propellant to deposit the antiperspirant composition onto an area of the skin. Aerosol antiperspirant products may transfer residue from the user to the fabric of a garment. This residue can be difficult to remove in regular laundering, and may decrease the consumer appeal of an aerosol antiperspirant product. Some antiperspirant products may employ silicas as suspending agents, which may suspend the active antiperspirant ingredient.

Accordingly, it is desirable to have an aerosol antiperspirant with decreased residue transfer from the axilla of the user to the fabric of a garment. In addition, it is desirable to maintain favorable skin feel properties, as these may also ameliorate consumer acceptance of such an aerosol antiperspirant. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An aerosol antiperspirant composition with decreased residue transfer to fabric comprises a propellant, an active antiperspirant ingredient, a number of silicas, and a number of polar emollients. The number of polar emollients interact with the silica to increase the viscosity of the antiperspirant composition upon volatilization of the propellant.

An aerosol antiperspirant composition with decreased residue transfer to fabric comprises a propellant, an active antiperspirant ingredient, a number of silicas, and a number of polar emollients, in which the number of polar emollients are configured to interact with the number of silicas to increase the viscosity of the antiperspirant composition upon volatilization of the propellant, wherein the viscosity of the composition following the volatilization of the propellant is within the range of 500 to 25,000 centipoise.

A method is provided for manufacturing an aerosol antiperspirant composition with high viscosity upon volatilization of the propellant. The method comprises adding a concentrated antiperspirant to a container, separately adding a thickening agent to the container, and mixing the thickening agent with the concentrated antiperspirant by adding a propellant to the container.

### BRIEF DESCRIPTION OF THE DRAWINGS

The present invention will hereinafter be described in conjunction with the following drawing figures, wherein like numerals denote like elements, and

FIG. 1 is a cross-sectional diagram of an exemplary container dispensing an aerosol antiperspirant composition according to the principles described herein;

FIG. 2 is a flowchart of a method for making an aerosol antiperspirant composition exhibiting reduced residue transfer.

FIG. 3 is a plot showing the relationship between a first silica concentration and viscosity in the presence or absence of a second silica in the absence of a polar emollient;

FIG. 4 is a plot showing the relationship between a first silica concentration and viscosity in the presence or absence of a second silica in the presence of a polar emollient;

FIG. 5 is a plot showing the relationship between a silica concentration and viscosity in the presence or absence of a polar emollient in the absence of a second silica;

FIG. 6 is a plot showing the relationship between a silica concentration and viscosity in the presence or absence of a polar emollient in the presence of a second silica;

FIG. 7 is a plot showing the relationship between the second silica concentration and viscosity in the presence or absence of a polar emollient in the absence of the first silica; and

FIG. 8 is a plot showing the relationship between the second silica concentration and viscosity in the presence or absence of a polar emollient in the presence of the first silica.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Antiperspirants may be compositions which reduce the amount of perspiration in an area of the skin to which they are applied. Antiperspirants may function by applying an active antiperspirant ingredient, which may dissolve in sweat and diffuse into the sweat glands, and may polymerize in combination with proteins to plug sweat ducts, which may bring about a decrease in the amount of perspiration in the area of the skin to which the antiperspirant was applied. An aerosol antiperspirant may use a propellant kept at high pressure to propel an antiperspirant composition out of a container to an area where the dispensing mechanism is directed. Upon release from the container, the propellant may volatilize, leaving the antiperspirant composition in the area treated with the aerosol antiperspirant composition. The remaining mixture of the antiperspirant composition may transfer residue onto clothing as the user gets dressed or thereafter.

Many factors may lead to consumer acceptance of an aerosol antiperspirant. For example, the amount of residue transferred to fabric may be a factor in the consumer acceptance of an aerosol antiperspirant composition. Additionally, the skin feel properties of an aerosol antiperspirant composition may also be valuable to consumer acceptance.

The efficacy of an antiperspirant product may also be a factor in consumer acceptance of an aerosol antiperspirant composition. Perspiration and odors emanating from perspiration may be both uncomfortable and embarrassing. For example, perspiration may make skin moist and sticky which may be uncomfortable. Perspiration may also be embarrassing for the individual, as it may transfer to a garment, and may be visible to others in a public setting, for example. Perspiration may also lead to wet spots, which may be uncomfortable as they may lead to chafing, and such wet spots may also be a source of embarrassment for the individual.

As perspiration may transfer to fabric and may lead to embarrassment, the residue from antiperspirants may also transfer to fabric. As with the perspiration, visible residue from antiperspirants may cause similar embarrassment to an individual, and may also lead to spotting or staining of a garment. Reductions in residue transfer to fabric may improve consumer acceptance of an aerosol antiperspirant composition, as the discomfort and embarrassment of both perspiration and antiperspirant residue may be avoided.

The viscosity of an aerosol antiperspirant composition following the volatilization of the propellant may be a factor in consumer acceptance. Increased viscosity following volatilization of the propellant may provide a decreased sensation of wetness upon application, and may also reduce the transfer of the antiperspirant from the axilla of the user to the fabric of a garment. This may in turn prevent the embarrassment associated with either perspiration or visible antiperspirant-based residue.

The principles described herein provide a composition for the reduction of residue transfer from an antiperspirant composition on the user to fabric. In other words, this could lead to less visible residue on garments associated with the use of antiperspirant compositions, while also maintaining comfort of the user with respect to a feeling of dryness. The principles described herein may also provide for improved skin feel properties of aerosol antiperspirant compositions.

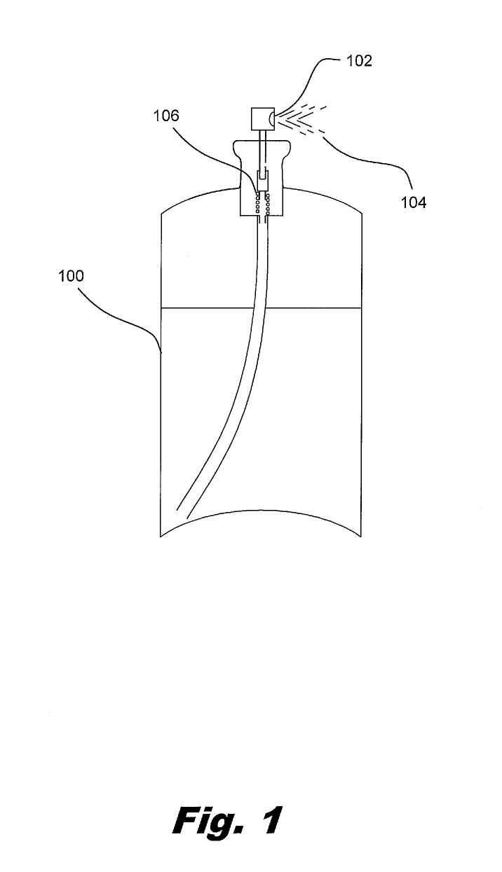

Turning now to the figures, FIG. 1 is a cross-sectional diagram of an exemplary container (100), dispensing an aerosol antiperspirant composition (104) according to the principles described herein. The container (100) may be equipped with an opening (102) capable of dispensing the aerosol antiperspirant composition (104). Such an opening (102) may be equipped with a spring (106), and may provide the ability to dispense the aerosol antiperspirant composition (104) to an area of the skin. For example, the opening (102) may provide the ability to dispense the aerosol antiperspirant composition (104) to the axilla of a user. In the example shown in FIG. 1, the container (100) is an aerosol canister. The container (100) shown in FIG. 1 is exemplary, and may not represent all types or shapes of containers for dispensing an aerosol antiperspirant composition (104). Another type of container (100) may be equipped with a different type of actuator, which includes the opening (102) and spring (106). Another type of opening (102) may be equipped with a short tube to localize application of the aerosol antiperspirant composition (104).

The aerosol antiperspirant composition (104) may include a propellant. A propellant may be any volatile ingredient which volatilizes on or before contact with the skin. A propellant may be a volatile hydrocarbon, fluorocarbon, hydrofluorocarbon, ether, another material which is gaseous at room temperature, or combinations thereof. Examples of propellants may include propane, n-butane, isobutane, dimethyl ether, methyl ethyl ether, nitrous oxide, carbon dioxide, air, nitrogen, hydrofluoroalkanes, or combinations thereof. Examples of hydrofluoroalkanes may include hydrofluorocarbon 152A (1,1-difluoro ethane), hydrofluoroalkane 134A (1,1,1,2-tetrafluoro ethane), hydrofluoroalkane 227 (1,1,1,2,3,3,3-heptafluoropropane), or combinations thereof. For example, a propellant may include n-butane and hydrofluorocarbon 152A.

The aerosol antiperspirant composition (104) may include an active antiperspirant ingredient. The active antiperspirant ingredient may be any ingredient that decreases perspiration, perspiration odors, perspiration wet spots, or combinations thereof. In some examples, the active antiperspirant agent may be a salt, which may dissolve in sweat and may diffuse into the eccrine gland duct and polymerize in combination with proteins to block the sweat ducts in the skin to which the aerosol antiperspirant composition (104) is applied. Thus, the active antiperspirant ingredient may prevent perspiration on the area of the skin to which the aerosol antiperspirant composition (104) is applied. Examples of active antiperspirant ingredients may include aluminum salts, aluminum zirconium salts, zirconium salts, or combinations thereof. Examples of aluminum salts may include aluminum halides (for example, aluminum chloride), aluminum chlorohydrate, aluminum hydroxyhalides, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, and combinations thereof. Examples of aluminum zirconium salts may include aluminum-zirconium octachlorohydrate, aluminum-zirconium trichlorohydrate, aluminum-zirconium tetrachlorohydrate, aluminum-zirconium pentachlorohydrate, aluminum-zirconium octachlorohydrate, aluminum-zirconium pentachlorohydrex glycine complex, aluminum zirconium octachlorohydrex glycine complex, and combinations thereof. Examples of zirconium salts may include zirconyl oxyhalides, zirconyl hydroxyhalides, zirconium chlorohydrate, and combinations thereof. For example, an active antiperspirant ingredient may include aluminum chlorohydrate.

The aerosol antiperspirant composition (104) may include a number of silicas. Silicas may be hydrophilic or hydrophobic, and may be modified or unmodified. Modifications to silica may include conjugation with hydrophobic groups to modulate the hydrophobicity of the silica. Fumed silica may be produced in a flame, and may have a high surface area to volume ratio. Silicas may be used in antiperspirant compositions as suspending agents. Silicas may also modulate the viscosity of an antiperspirant composition. Examples of silicas may include Aeroperl® and Aerosil® silicas, which are manufactured by Evonik Industries, with headquarters in Essen, Germany, as well as Cab-O-Sil® silicas, which are manufactured by Cabot Corporation, with headquarters in Boston, Mass. Exemplary Aeroperl® and Aerosil® silicas may include Aeroperl® 300/30, Aerosil® 200, Aerosil® 300, Aerosil® 380, Aerosil® R 202, Aerosil® R 805, Aerosil® R 812, Aerosil® R 812 S, Aerosil® R 972, Aerosil® R 972 V, Aerosil® R 974, Aerosil® R 974 V. Exemplary Cab-O-Sil® silicas may include Cab-O-Sil® TS-610, Cab-O-Sil® TS-530, Cab-O-Sil® TS 622. The aerosol antiperspirant composition (104) may also employ other fumed silicas, and any combinations thereof. For example, the number of silicas may comprise Aerosil® 300 and Aerosil® R 972 V. In another example, the number of silicas may comprise Aerosil® R 812. Throughout this disclosure specific reference is made to specific Aeroperl® and Aerosil® silicas, however, the systems and methods disclosed herein may be implemented with any type of silica.

The aerosol antiperspirant composition (104) may include a number of polar emollients. An emollient may be a compound which helps to maintain the soft, smooth and pliable appearance of skin. Emollients may function by their ability to remain on the skin surface or in the stratum corneum to act as lubricants, reduce flaking, and to improve the skin's appearance. Polar emollients may have a dipole moment, which may be characterized by measuring their dielectric constant. A dielectric constant may depend on the hydrocarbon chain length and extent of branching, as well as the presence of certain functional groups, such as oxygen, which may affect the local concentration of charge on a molecule. Because oxygen atoms may increase polarity, esters may have higher dielectric constants than hydrocarbons. A polar emollient may interact with silica to modulate the viscosity of the antiperspirant composition, which may be noticeable to the user upon volatilization of the propellant. In some embodiments, the polar emollient may comprise less than 1.0 weight percent of the aerosol antiperspirant composition (104), for example less than 0.8 weight percent of the aerosol antiperspirant composition (104). A polar emollient may be selected to increase the viscosity of an aerosol concentrate on skin after application.

An example of a polar emollient which may be used in the aerosol antiperspirant composition (104) is triethyl citrate. Triethyl citrate may have three ester groups, and other polar emollients may have a number of ester groups as well. An ester, shown below, may include a carbonyl adjacent to an ether linkage.

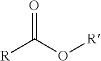

In the above example of an ester, R and R′ may be any alkyl or aryl group, including linear, branched, cyclic, aromatic groups, or combinations thereof. In addition to hydrocarbon substituents, R and R′ may also include additional functionalities, including halides, esters, ethers, alcohols, aldehydes, ketones, amides, amines, thiols, thioethers, thioesters, and combinations thereof.

The combination of polar emollient and silica in the aerosol antiperspirant composition (104) may increase the viscosity of the composition. The increase in viscosity of the aerosol antiperspirant composition (104) may be modulated by adjustment of the relative amounts of the components of the aerosol antiperspirant composition (104). The viscosity may be a function of the concentration of the number of silicas, as well as the relative concentrations of the silicas present in the aerosol antiperspirant composition (104). The viscosity may also be a function of the concentration of the polar emollient, and the relative concentrations of the polar emollient to the number of silicas present in the composition.

For example, if the number of silicas comprise Aerosil® R972 V and Aerosil® 300, and the polar emollient comprises triethyl citrate, the viscosity was found to be a function of these three components. The viscosity may be modulated according to the formula,

\sqrt{V}=7.2+12.7(A₁)+31.5(A₂)+1.2(E)+118.9(A₁)(A₂)+67.7(A₁)(E) Equation (1)

wherein V may be the viscosity of the aerosol antiperspirant composition (104) after the propellant has volatilized (or in the absence of propellant), expressed in units of centipoise. A₁ may be the concentration of the first silica, Aerosil® R 972 V for example, A₂ may be the concentration of the second silica, Aerosil® 300 for example, and E may be the concentration of the polar emollient, triethyl citrate. In Equation (1), A₁, A₂, and E may be expressed in units of weight percent.

Equation (1) may highlight the non-obvious nature of the synergy between a number of silicas and the polar emollient with respect to the change to viscosity. While each silica and polar emollient may have some bearing on the viscosity of the aerosol antiperspirant composition (104), the combined term (A₁)(E) may have a much larger coefficient, which may be indicative of a synergistic effect on the viscosity which results from the combination of the silica with a polar emollient, which polar emollient in this example is triethyl citrate.

Increasing the viscosity of the aerosol antiperspirant composition (104) may decrease the amount of visible residue that is transferred to a garment. This reduction in visible residue on a garment may alleviate the embarrassment of both perspiration and antiperspirant residue. Thus, it is desirable for an aerosol antiperspirant composition (104) to have an increased viscosity. In certain embodiments, the viscosity may be increased to within the range of 1,000 to 22,000 centipoise upon volatilization of the propellant. However, increasing the viscosity of an aerosol antiperspirant composition (104) may make such a composition difficult to dispense from an aerosol container (100) in the desired spray pattern.

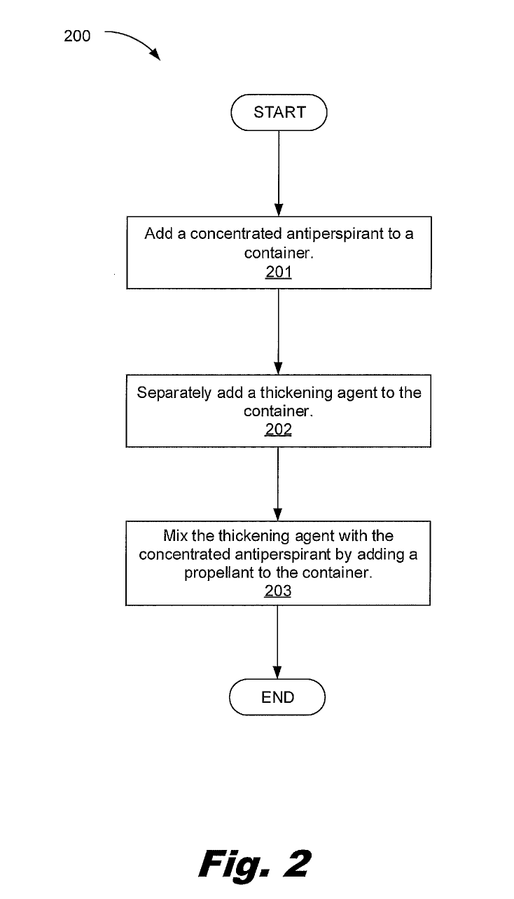

Any increase in viscosity may be delayed by the addition of propellant prior to, or simultaneous with, mixing of the components of the aerosol antiperspirant composition (104), for example mixing of the silica with the emollient. FIG. 2 shows a flowchart which diagrams a method for making an aerosol antiperspirant composition (FIG. 1, 104) which delays the viscosity increase. As shown in FIG. 2, a concentrated antiperspirant may be added (block 201) to a container, and a thickening agent may be added (block 202) to a container separately. The thickening agent and the concentrated antiperspirant may be mixed (block 203) by adding a propellant to the container. The mixing of the silicas with the emollient may bring about an increase in viscosity, but this interaction may be delayed by the dilution with propellant. The method diagramed in FIG. 2 is exemplary, and may not represent all methods for delaying the increase in viscosity of the aerosol antiperspirant composition (FIG. 1, 104). For example, the active antiperspirant ingredient may be mixed with ingredients other than the emollient and propellant, and this mixture may be added to an aerosol container (FIG. 1, 100). The emollient may then be added, and then the aerosol container (FIG. 1, 100) may be sealed. The propellant may then be added, which may mix the emollient with the other ingredients. Further mixing may be accomplished by manually agitating the aerosol container (FIG. 1, 100). In another example, the emollient may be mixed with ingredients other than the silica or propellant, and this mixture may be added to an aerosol container (FIG. 1, 100). The silica may then be added, and then the aerosol container (FIG. 1, 100) may be sealed. The propellant may then be added, which may mix the silica with the other ingredients, including the emollient. As with the previous example, further mixing may be accomplished by manual agitation of the sealed aerosol container (FIG. 1, 100). In this example, the thickening agent may include the number of silicas and a polar emollient, or combinations thereof. The mixing of the number of silicas with the number of polar emollients may be delayed in a similar manner as in the previous example.

Delaying the increase in the viscosity of the composition may allow for facile dispensing of the aerosol antiperspirant composition (FIG. 1, 104) from the container (FIG. 1, 100) through the opening (FIG. 1, 102), whereupon the viscosity may increase upon volatilization of the propellant.

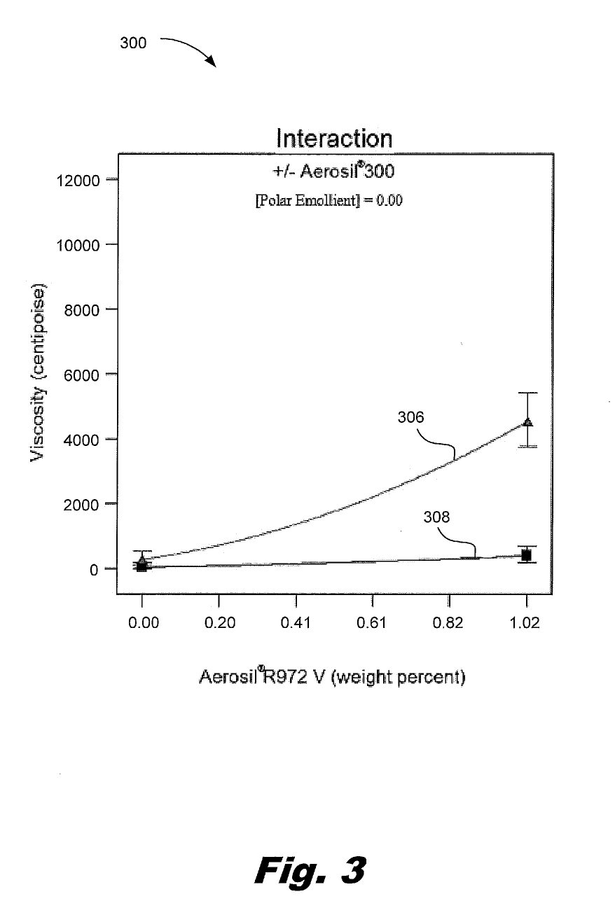

The dependence of the viscosity of the aerosol antiperspirant composition (FIG. 1, 104) on the synergistic interactions between the number of silicas and the polar emollient may also be shown in FIGS. 3, 4, 5, 6, 7, and 8. The data shown in FIGS. 3, 4, 5, 6, 7, and 8 may be following the volatilization of the propellant, and may represent the viscosity that may be sensed by the user in their axilla.

FIG. 3 is an interaction plot (300) which may show the change in viscosity which results from changes to the concentration of one of the silicas, in this case Aerosil® R 972 V. The data for FIG. 3 may be from an aerosol antiperspirant composition (FIG. 1, 104) lacking a polar emollient. The upper curve (306) may represent the change in viscosity which results from changing the concentration of Aerosil® R 972 V when the concentration of Aerosil® 300 is held constant at 0.31 weight percent of the aerosol antiperspirant composition (FIG. 1, 104). Similarly, the lower curve (308) may represent the change in viscosity which results from changes to the concentration of Aerosil® R 972 V when the aerosol antiperspirant composition (FIG. 1, 104) does not contain Aerosil® 300. In FIG. 3, viscosity may be measured in centipoise, and the concentration of Aerosil® R 972 V may be measured as the weight percentage of the aerosol antiperspirant composition (FIG. 1, 104).

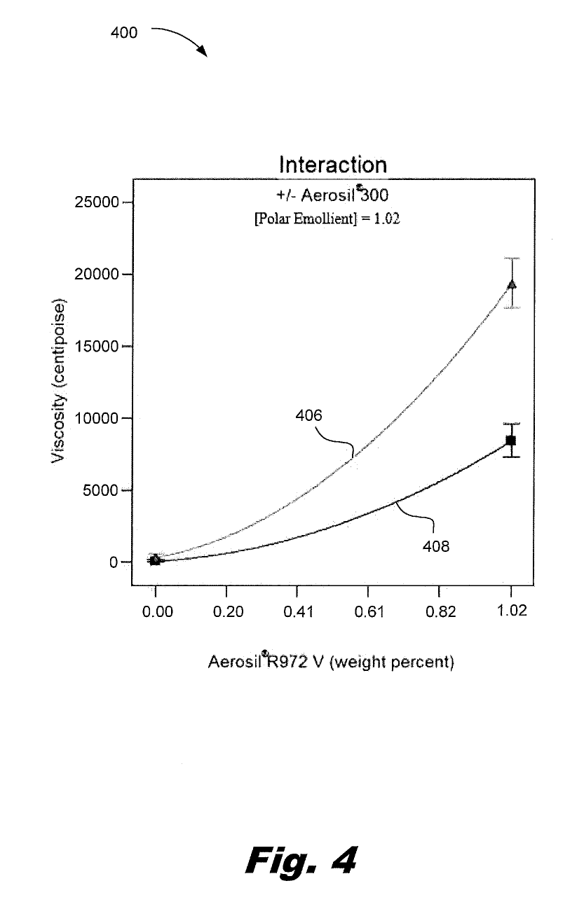

FIG. 4 is an interaction plot (400) which may show the change in viscosity which results from changes to the concentration of one of the silicas, in this case Aerosil® R 972 V. The data for FIG. 4 may be from an aerosol antiperspirant composition (FIG. 1, 104) which contains 1.02 weight percent of triethyl citrate, which may be a polar emollient. The upper curve (406) may represent the change in viscosity which results from changing the concentration of Aerosil® R 972 V when the concentration of Aerosil® 300 is held constant at 0.31 weight percent of the aerosol antiperspirant composition (FIG. 1, 104). Similarly, the lower curve (408) may represent the change in viscosity which results from changes to the concentration of Aerosil® R 972 V when the aerosol antiperspirant composition (FIG. 1, 104) does not contain Aerosil® 300. In FIG. 4, viscosity may be measured in centipoise, and the concentration of Aerosil® R 972 V may be measured as the weight percentage of the aerosol antiperspirant composition (FIG. 1, 104).

The synergism that may exist between the polar emollient and the number of silicas may be visible in the differences between FIGS. 3 and 4. As indicated in FIG. 4, the presence of a polar emollient may increase the extent of the viscosity increase when increasing concentrations of one of a number of silicas as compared to the absence of a polar emollient in similar circumstances as indicated in FIG. 3. This synergistic interaction may be represented in Equation (1) by the factor (A/)(E).

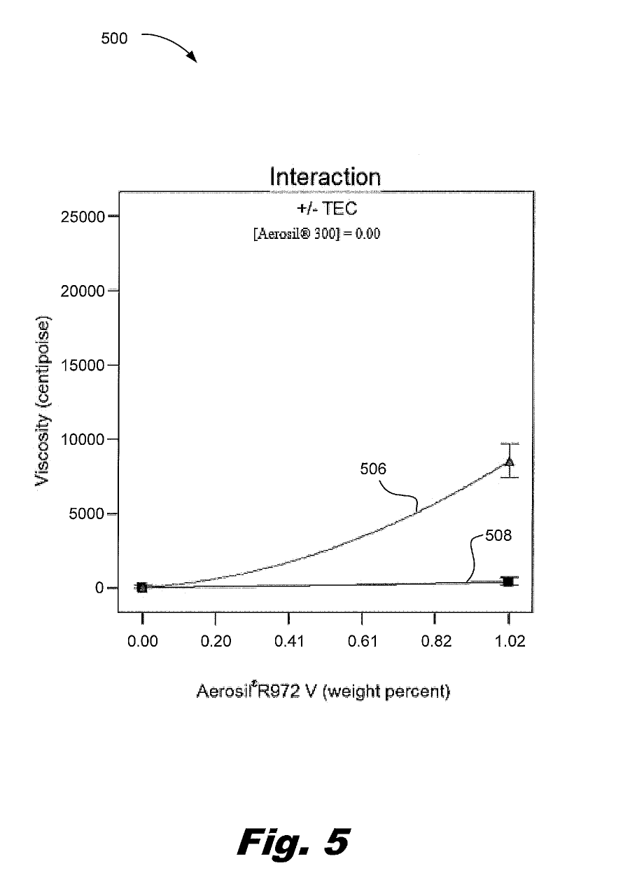

FIG. 5 is an interaction plot (500) which may show the change in viscosity which results from changes to the concentration of one of the silicas, in this case Aerosil® R 972 V. The data for FIG. 5 may be from an aerosol antiperspirant composition (FIG. 1, 104) which may use Aerosil® R 972 V as the silica. The upper curve (506) may represent the change in viscosity which results from changing the concentration of Aerosil® R 972 V when the concentration of triethyl citrate, which may be a polar emollient, is held constant at 1.02 weight percent of the aerosol antiperspirant composition (FIG. 1, 104). Similarly, the lower curve (508) may represent the change in viscosity which results from changes to the concentration of Aerosil® R 972 V when the aerosol antiperspirant composition (FIG. 1, 104) does not contain a polar emollient. In FIG. 5, viscosity may be measured in centipoise, and the concentration of Aerosil® R 972 V may be measured as the weight percentage of the aerosol antiperspirant composition (FIG. 1, 104).

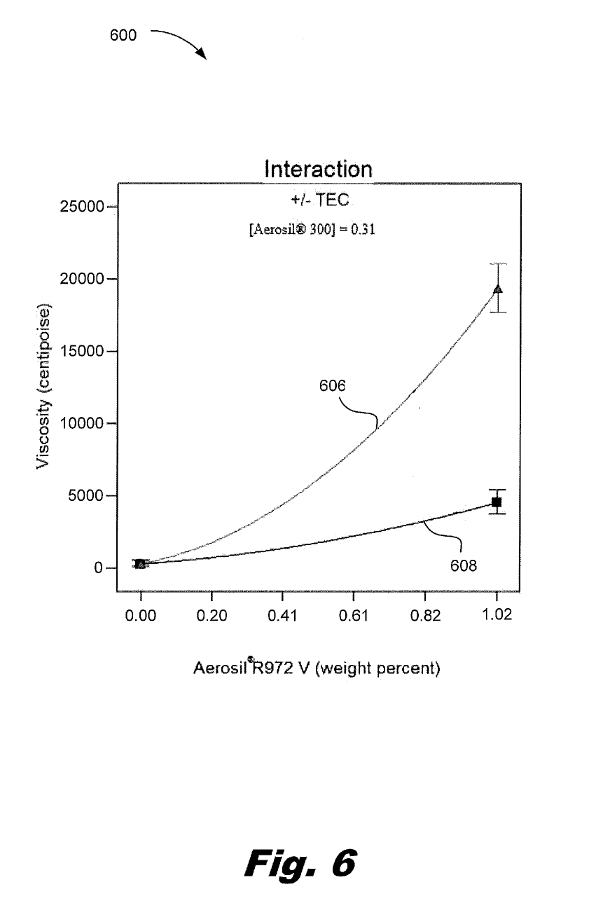

FIG. 6 is an interaction plot (600) which may show the change in viscosity which results from changes to the concentration of one of the silicas, in this case Aerosil® R 972 V. The data for FIG. 6 may be from an aerosol antiperspirant composition (FIG. 1, 104) which comprises both Aerosil® R 972 V and Aerosil® 300, wherein the concentration of Aerosil® 300 is held constant at 0.31 weight percent. The upper curve (606) may represent the change in viscosity which results from changing the concentration of Aerosil® R 972 V when the concentration of triethyl citrate, which may be a polar emollient, is held constant at 1.02 weight percent of the aerosol antiperspirant composition (FIG. 1, 104). Similarly, the lower curve (608) may represent the change in viscosity which results from changes to the concentration of Aerosil® R 972 V when the aerosol antiperspirant composition (FIG. 1, 104) does not contain a polar emollient. In FIG. 6, viscosity may be measured in centipoise, and the concentration of Aerosil® R 972 V may be measured as the weight percentage of the aerosol antiperspirant composition (FIG. 1, 104).

The synergism that may exist between the polar emollient and the number of silicas may be visible in the differences between FIGS. 5 and 6. As indicated in FIG. 6, the presence of a second of a number of silicas may increase the extent of the viscosity increase when increasing concentrations of one of a number of silicas as compared to the absence of a second silica in similar circumstances, as indicated in FIG. 5. FIGS. 5 and 6 may also emphasize the synergistic relationship between a silica used as the independent variable and the polar emollient (triangles, 506/606) as compared to those lacking the polar emollient (squares, 508/608), with respect to the viscosity of the aerosol antiperspirant composition (104). The differences between FIGS. 5 and 6 may emphasize the synergy between the silicas, which may be represented in Equation (1) by the factor (A₁)(A₂).

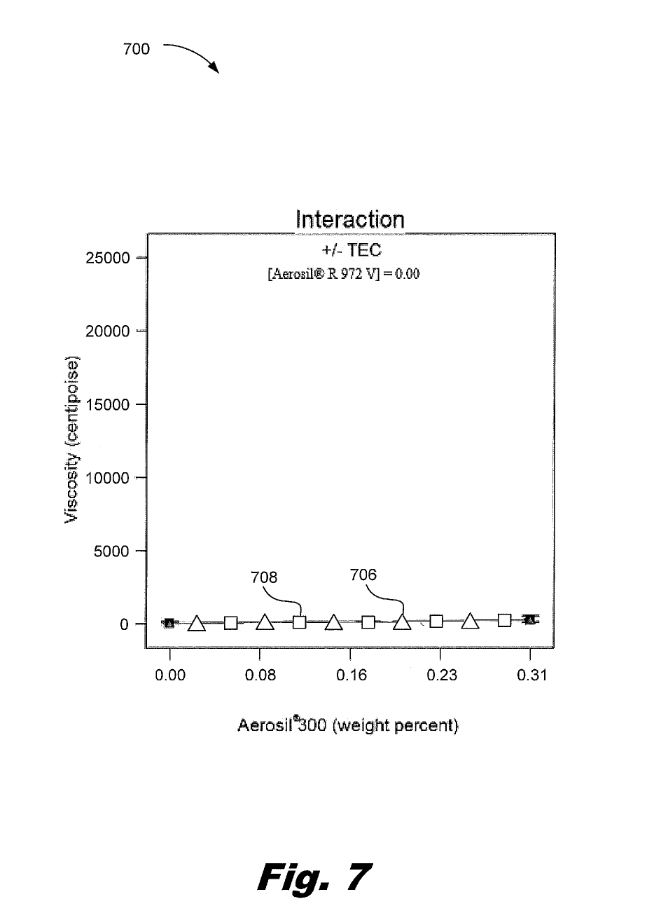

FIG. 7 is an interaction plot (700) which may show the change in viscosity which results from changes to the concentration of one of the silicas, in this case Aerosil® 300. The data for FIG. 7 may be from an aerosol antiperspirant composition (FIG. 1, 104) which may use Aerosil® 300 as the only one of the number of silicas. It should be noted that FIG. 7 depicts two curves. A first curve (706) indicated by triangle markers, may represent the change in viscosity which results from changing the concentration of Aerosil® 300 when the concentration of triethyl citrate, which may be a polar emollient, is held constant at 1.02 weight percent of the aerosol antiperspirant composition (FIG. 1, 104). A second curve (708) indicated by square markers may represent the change in viscosity which results from changes to the concentration of Aerosil® 300 when the aerosol antiperspirant composition (FIG. 1, 104) does not contain a polar emollient. In FIG. 7, viscosity may be measured in centipoise, and the concentration of Aerosil® 300 may be measured as the weight percentage of the aerosol antiperspirant composition (FIG. 1, 104).

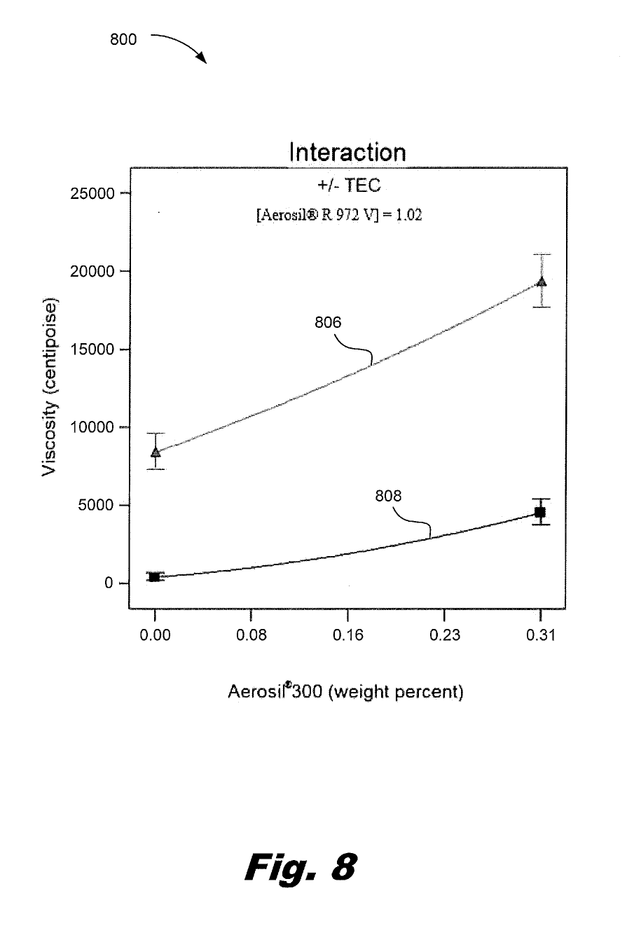

FIG. 8 is an interaction plot (800) which may show the change in viscosity which results from changes to the concentration of one of the silicas, in this case Aerosil® 300. The data for FIG. 8 may be from an aerosol antiperspirant composition (FIG. 1, 104) which comprises both Aerosil® R 972 V and Aerosil® 300, wherein the concentration of Aerosil® R 972 V is held constant at 1.02 weight percent. The upper curve (806) may represent the change in viscosity which results from changing the concentration of Aerosil® 300 when the concentration of triethyl citrate, which may be a polar emollient, is held constant at 1.02 weight percent of the aerosol antiperspirant composition (FIG. 1, 104). Similarly, the lower curve (808) may represent the change in viscosity which results from changes to the concentration of Aerosil® 300 when the aerosol antiperspirant composition (FIG. 1, 104) does not contain a polar emollient. In FIG. 8, viscosity may be measured in centipoise, and the concentration of Aerosil® 300 may be measured as the weight percentage of the aerosol antiperspirant composition (FIG. 1, 104).

By comparison of FIGS. 7 and 8, the synergistic relationship that may exist between the polar emollient and certain silicas which comprise the number of silicas may be apparent. Furthermore, the distinctions between FIGS. 7 and 8 may highlight the unexpected nature of the synergism between the number of silicas and the polar emollient, as the data in these two figures may differ only in the amount of one of the number of silicas which may be present in the aerosol antiperspirant composition (FIG. 1, 104).

As the viscosity of the aerosol antiperspirant composition (FIG. 1, 104) following volatilization of the propellant may be valuable to the application aesthetics of the composition, the ability to effectively modulate the viscosity by including silicas and polar emollients in appropriate amounts may be extraordinarily valuable, and may significantly increase the consumer acceptance of the aerosol antiperspirant composition (FIG. 1, 104). Additionally, as the viscosity of an aerosol antiperspirant composition (FIG. 1, 104) may affect its propensity to transfer residue from the axilla of the user to fabric that may be in contact or close proximity to the axilla of the user, and a higher viscosity may transfer less residue, increasing the viscosity of the composition may decrease the residue transfer from the skin of the user to fabric. Decreasing the amount of residue transfer may further ameliorate the consumer acceptance of the aerosol antiperspirant composition (FIG. 1, 104).

The aerosol antiperspirant composition (FIG. 1, 104) may further comprise a number of siloxanes. Siloxanes may be compounds which have an Si—O—Si linkage, and may be linear, branched, cyclic, or combinations thereof. Siloxanes may be emollients. Examples of siloxanes include octamethylcyclotetrasiloxane, decamethylcyclopentasiloxane, dodecamethylcyclohexasiloxane, decamethyltetrasiloxane, dodecamethylpentasiloxane, similar siloxanes with larger alkyl groups (for example, ethyl, propyl, isopropyl, butyl, isobutyl, and tert-butyl), similar siloxanes with cyclic or aromatic groups bound to the silicon atoms, other polysiloxanes, or combinations thereof.

The aerosol antiperspirant composition (FIG. 1, 104) may further comprise additional components, which may include fragrance, talc, suspension agents, or combinations thereof. These components may be added to improve the scent, skin feel properties, or consumer acceptance of the aerosol antiperspirant composition (FIG. 1, 104).

Table (1) below may present exemplary amounts of each component of the aerosol antiperspirant composition (FIG. 1, 104). The composition in Table (1) may be suitable for use as an aerosol antiperspirant product to reduce perspiration, as well as to reduce the residue transfer to fabric. In the disclosed formulation, aluminum chlorohydrate may be used as the active antiperspirant ingredient, Aerosil® R 972 V and Aerosil® 300 may be used as the number of silicas, cyclopentasiloxane and cyclotetrasiloxane may be a number of siloxanes in the composition, the number of polar emollients in the composition may comprise triethyl citrate, and a mixture of 66% hydroflourocarbon 152A with 34% butane may be used as the propellant. In the table below, the active antiperspirant ingredient, the number of silicas, talc and the number of siloxanes may comprise the concentrated antiperspirant. The thickener may comprise the number of polar emollients and fragrance. Thus, the product may be assembled by preparing the concentrated antiperspirant, adding this to a container, separately adding the thickener to the container, then adding the propellant to the container to mix the concentrated antiperspirant with the thickener.

TABLE (1)

| Ingredient | Amount (% weight) |
|---|---|
| Aluminum chlorohydrate | 10.12 |
| Aerosil ® R 972 V | 0.80 |
| Aerosil ® 300 | 0.20 |
| Talc | Trace |
| Cyclopentasiloxane | 14.49 |
| Cyclotetrasiloxane | 14.49 |
| Triethyl Citrate | 0.70 |
| 66% Hydrofluorocarbon 152A/34% Butane | Rest |

As described above, the aerosol antiperspirant composition (FIG. 1, 104) may reduce the amount of white residue deposited on a fabric. For example, the white residue resultant on a fabric treated with an aerosol antiperspirant composition (FIG. 1, 104) may be compared to the white residue resultant on a fabric treated with a control composition. The control composition may include the following ingredients in the following amounts:

TABLE (2)

| Ingredient | Amount (% weight) |
|---|---|
| Aluminum chlorohydrate | 10.12 |
| Aerosil ® R 972 V | 1.00 |
| Aerosil ® 300 | 0.25 |
| Talc | Trace |
| Cyclopentasiloxane | 14.49 |
| Cyclotetrasiloxane | 14.49 |
| Triethyl Citrate | 0.00 |
| 66% Hydrofluorocarbon 152A/34% Butane | Rest |

Table (3) may indicate the detected white residue on fabrics treated with the aerosol antiperspirant composition (FIG. 1, 104) and the control composition. For example, a fabric may have been treated with either 1) the aerosol antiperspirant composition (FIG. 1, 104) or 2) the control composition. Measurements of white residue may have been taken at different time intervals t0, t15, t60, and t24h which represent time intervals of 0 minutes, 15 minutes, 60 minutes and 24 hours. The white residue may then be measured on each fabric at each time interval and a numerical value assigned to the amount of detected white residue with 0 representing zero staining and 4 representing intense staining.

TABLE (3)

| | t0 | t15 | t60 | t24 h |
|---|---|---|---|---|
| Aerosol Antiperspirant Composition | 0.20 | 2.00 | 3.00 | 3.50 |
| (FIG. 1, 104) | | | | |
| Control Composition | 1.75 | 3.00 | 4.00 | 5.00 |

The values indicated in Table (3) may represent mean values of multiple measurements taken at the corresponding time intervals. As indicated in Table (3), the aerosol antiperspirant composition (FIG. 1, 104) having triethyl citrate may reduce the amount of white residue present as compared to the control composition as indicated by the lower scores at each interval.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. An aerosol antiperspirant composition with decreased residue transfer to fabric, comprising:
a propellant;
an active antiperspirant ingredient;
at one hydrophilic silica
at least one hydrophobic silica; and
triethyl citrate, in which the triethyl citrate interacts with the silicas to increase the viscosity of the antiperspirant composition upon volatilization of the propellant.

2. (canceled)

3. (canceled)

4. The aerosol antiperspirant composition of claim 1, wherein the triethyl citrate comprises less than 1.0 weight percent of the composition.

5. The aerosol antiperspirant composition of claim 1, wherein the triethyl citrate comprises less than 0.8 weight percent of the composition.

6. The aerosol antiperspirant composition of claim 1, further comprising a siloxane.

7. The aerosol antiperspirant composition of claim 1, further comprising talc, fragrance, or combinations thereof.

8. An aerosol antiperspirant composition with decreased residue transfer to fabric, comprising:
a propellant;
an active antiperspirant ingredient;
at one hydrophilic silica at least one hydrophobic silica; and
triethyl citrate, in which the triethyl citrate interacts with the silicas to increase the viscosity of the antiperspirant composition upon volatilization of the propellant,
wherein the viscosity of the composition following the volatilization of the propellant is within the range of 500 to 25,000 centipoise.

9. The aerosol antiperspirant composition of claim 8, wherein the viscosity of the composition following the volatilization of the propellant is within the range of 1,000 to 22,000 centipoise.

10. The aerosol antiperspirant composition of claim 8, wherein the viscosity of the composition before the volatilization of the propellant is within the range of 10 to 500 centipoise.

11. (canceled)

12. (canceled)

13. The aerosol antiperspirant composition of claim 13, wherein the triethyl citrate comprises less than 1.0 weight percent of the composition.

14. (canceled)

15. A method for making an aerosol antiperspirant composition, comprising:
adding a concentrated antiperspirant to a container;
separately adding a thickening agent to the container; and
mixing the thickening agent with the concentrated antiperspirant by adding a propellant to the container.

16. The method of claim 15, wherein the thickening agent comprises a number of polar emollients.

17. The method of claim 16, wherein the number of polar emollients comprise triethyl citrate.

18. The method of claim 15, further comprising adding a number of silicas to the concentrated antiperspirant.

19. The method of claim 15, further comprising adding a number of silicas to the container.

20. The method of claim 15, wherein
the concentrated antiperspirant is mixed with the number of silicas, which mixture is added first to a container;
the thickening agent is added to the container second;
the container is sealed; and
propellant is added to the container, which mixes the thickening agent with the silicas and the concentrated antiperspirant.
